(19) United States
(12) Patent Application Publication
(10) Pub. No.: US 2012/0029531 A1
(43) Pub. Date: Feb. 2, 2012

(54) SUTURE CUT-OFF STRUCTURE FOR SUBCUTANEOUS STITCHING OPERATION

(76) Inventor: YEN-YUE LIN, Taipei (TW)

(21) Appl. No.: 13/270,237

(22) Filed: Oct. 11, 2011

Related U.S. Application Data
(63) Continuation-in-part of application No. 11/970,536, filed on Jan. 8, 2008.

(51) Int. Cl. A61B 17/04

(52) U.S. Cl. 606/138

(57) ABSTRACT

A suture cut-off structure for subcutaneous stitching operation includes a forceps and a cutter blade arranged at one end of the forceps. The cutter blade has an end forming two rims, which correspond to each other and have the same length. Each rim has a contour forming a curve section. The rims define therebetween a cutting trough in which a cutting edge is formed. The suture is receivable into the cutting trough to allow the suture to be cut off through a forward movement of the cutting edge. With the rims being positionable on and depressing down patient's skin, cutting of the suture and at the same time depressing down the patient's skin can be simultaneously done with a single hand The cutting edge is located inside the cutting trough of the rims and is thus isolated so that undesirably hurting the patient's skin can be avoided.

### CROSS-REFERENCE TO RELATED APPLICATION

This is a continuation-in-part of the co-pending patent application Ser. No. 11/970,536, owned by the same applicant.

### TECHNICAL FIELD OF THE INVENTION

The present invention generally relates to a suture cut-off structure for surgical operations, and more particularly to a suture cut-off structure for subcutaneous stitching operations that efficiently cuts off a surgical suture, comfortably and stably depresses human skin, and makes no undesired damage to human skin.

### DESCRIPTION OF THE PRIOR ART

When a surgeon performs a stitching closure operation, the surgeon uses one hand to hold a pair of forceps for grasping skin around a wound and uses another hand to operate a needle holder in order to perform closure of wound in a stitch-by-stitch manner with the aid of the needle holder. However, when the surgeon carries out a stitching operation, each stitch must be ended with knotting, following by suture severing with a pair of scissors, and only after that, the following stitches can be subsequently made. During the stitching process, it is always troublesome that the surgeon must empty one hand to hold and operate the scissors for cutting of the suture after each stitching is made, and then the following stitch can be subsequently carried out. Alternately, an assistant may be available for helping cutting off the suture. However, it is equally troublesome for both the surgeon and the assistant operating the scissors to cut off the suture and this takes labor and is uneconomic.

In addition, since the scissors have sharp tips that are not provided with protection means, it is quite likely that the patient's skin is unexpectedly hurt in using the scissors to cut off the suture. Further, in the performance of subcutaneous stitching operation, besides the troubles of frequently changing forceps and scissors, a surgeon also needs to depress down the skin around the wound first and then the suture located under the skin can be tied and pulled out to allow a portion above the tying location to be cut off with the scissors. Holding down the skin around the wound often causes discomfort of the patient and may accidently hurt the skin.

In view of the above discussed shortcomings, the present invention aims to provide a solution to overcome such problems.

### SUMMARY OF THE INVENTION

An objective of the present invention is to provide a suture cut-off structure for subcutaneous stitching operation that can be efficiently and operate and does not cause undesired damage to patents' skin.

To achieve the above objective, the present invention provides a suture cut-off structure for subcutaneous stitching operation, which comprises a forceps and a cutter blade arranged at one end of the forceps. The forceps has an end forming a stalk-like cutter holder in which a slit is formed. The cutter blade forms a through hole, which comprises a relatively-wide first retention section and a relatively-narrow second retention section. The first retention section is fit over a body of the stalk-like cutter holder, while the second retention section is fit into the slit of the stalk-like cutter holder so as to securely retain the cutter blade on the stalk-like cutter holder, thereby coupling the cutter blade and the stalk-like cutter holder together. The cutter blade has an end forming two rims, which correspond to each other and have substantially the same length. Each of the rims has a contour forming a curve section. The rims define a cutting trough therebetween. A cutting edge is formed in the cutting trough. The suture is receivable into the cutting trough to allow the suture to be cut off through a forward movement of the cutting edge, and with the rims being positionable on and depressing down patient's skin, cutting of the suture and at the same time depressing down the patient's skin can be simultaneously done with a single hand. The cutting edge is located inside the cutting trough of the rims and is thus isolated so that undesirably hurting the patient's skin can be avoided. With the rims having the same length and showing the curve section, comfortably and stably holding down the patient's skin can be done without causing undesired hurting, thereby allowing efficiently cutting of suture in a subcutaneous stitching operation.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is an exploded view of the present invention.

FIG. 2 is a plan view, in an exploded form, of a portion of forceps and a cutter blade according to the present invention.

FIG. 3 is a perspective view of the present invention.

FIG. 4 is a plan view showing the forceps and the cutter blade according to the present invention coupled to each other.

FIGS. 5-8 are schematic views demonstrating a subcutaneous stitching operation according to the present invention.

FIG. 9 demonstrates cutting off suture according to the present invention applied to a subcutaneous stitching operation.

FIG. 10 is a side elevational view illustrating cutting off suture as shown in FIG. 9.

FIG. 11 is a schematic view illustrating a knot tied on a suture is located under the patient's skin after the suture is cut off in a subcutaneous stitching operation.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

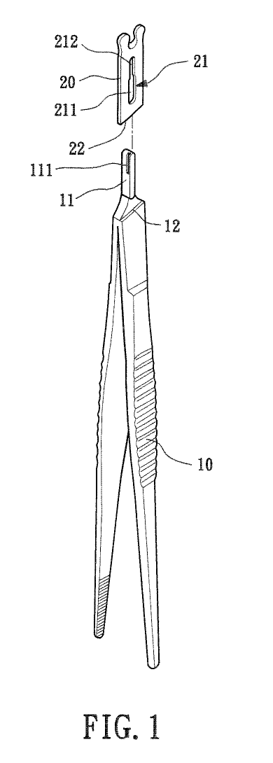

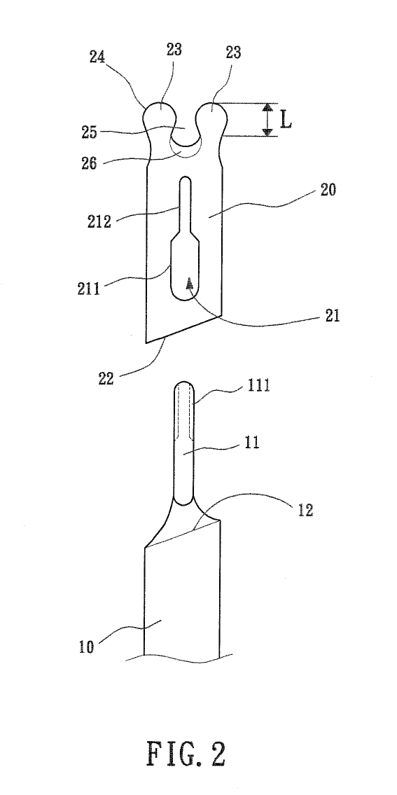

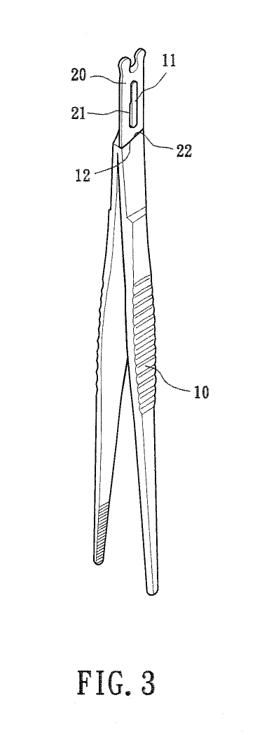

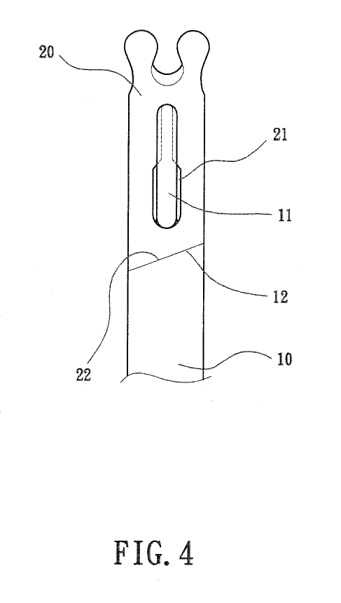

Referring to FIGS. 1, 2, 3, and 4, a preferred embodiment according to the present invention is shown, which comprises a forceps 10 and a cutter blade 20 arranged at one end of the forceps 10, whereby in an attempt to cut off a suture in performing a stitching operation, the forceps 10 and the cutter blade 20 can be switchably used in order to efficiently cut off the suture without causing undesired damage to the patient's skin. The present invention finds a particular application to subcutaneous stitching operations.

The forceps 10 has an end forming a stalk-like cutter holder 11 in which a slit 111 is formed. The forceps 10 also forms an inclined step 12 below the stalk-like cutter holder 11.

The cutter blade 20 forms a through hole 21, and the through hole 21 comprise a first retention section 211 that is relatively wide and a second retention section 212 that is relatively narrow. The first retention section 211 is fit over a body of the stalk-like cutter holder 11, while the second retention section 212 is fit into the slit 111 of the stalk-like cutter holder 11, so as to securely retain the cutter blade 20 on the stalk-like cutter holder 11, thereby coupling the cutter blade 20 and the stalk-like cutter holder 11 together. The cutter blade 20 forms a support section 22 corresponding to the step 12 of the forceps 10, whereby with the interaction between the step 12 and the support section 22, the cutter blade 20 is securely held on the stalk-like cutter holder 11. The cutter blade 20 has an end forming two rims 23, which correspond to each other and have substantially the same length L. Each of the rims 23 has a contour forming a curve section 24. The rims 23 define a cutting trough 25 therebetween, and a cutting edge 26 is formed in the cutting trough.

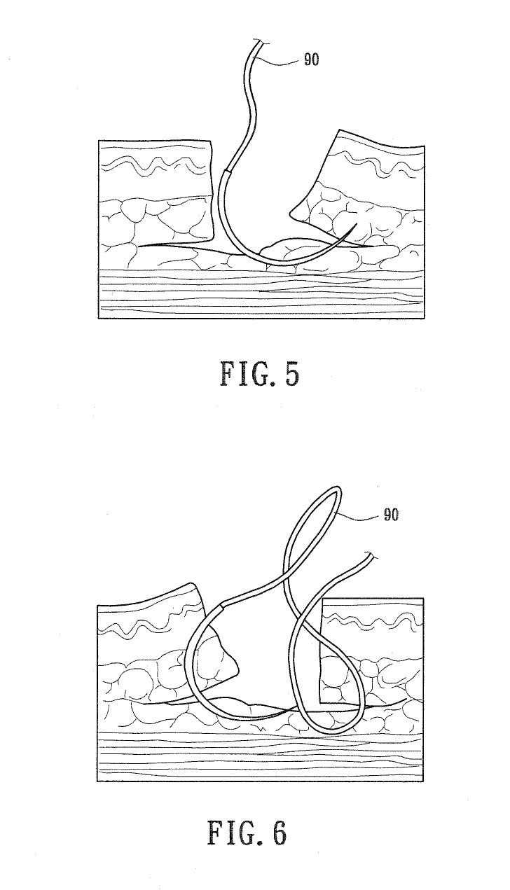

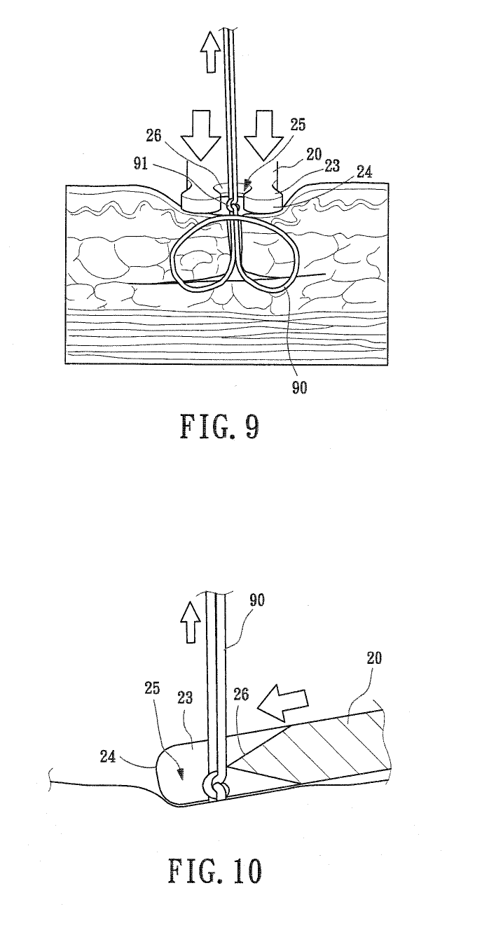

Referring to FIGS. 5, 6, 7, and 8, a subcutaneous stitching operation is demonstrated. FIG. 10 illustrates a knot is tied on a suture 90 and the knot 91 of the suture 90 is located under the skin of the patient.

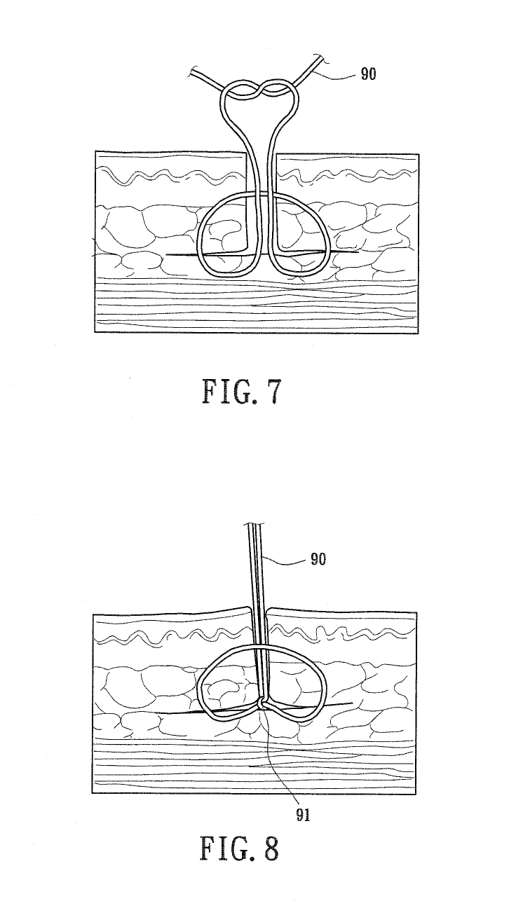

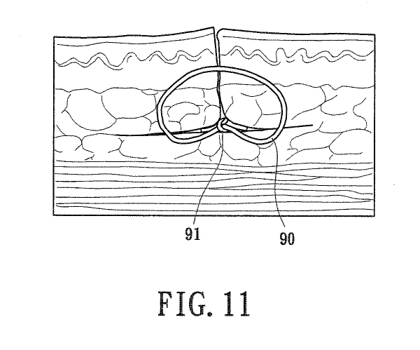

Referring to FIGS. 9, 10, and 11, the present invention allows a surgeon to directly switch the forceps 10 and the cutter blade 20 for operation needs and is also helpful depressing and holding down the patient's skin around the wound with the rims 23 of he cutter blade 20 after the suture 90 used in the subcutaneous stitching operation has been knotted in order to allow the knot 91 of the suture 90 that is located under the skin to be pulled out of the skin, and then through pushing forwards the cutter blade 20, the knotted the suture 90 can be received into the cutting trough 25 and further pushing forwards would cause the cutting edge 21 to cut off an extra portion of the suture 90 that is located above the knot 91. As such, when a surgeon is performing a subcutaneous stitching operation, it does not need to use an independent pair of scissors with seeking for other people's help to cut off the suture 90.

It is noted here that since the rims 23 of the cutter blade 20 can be placed on and depress down the patient's skin, a surgeon is allowed to cut off the suture and at the same time depress down the patient's skin, both being done with a single hand. Further, since the cutting edge 26 is located inside the cutting trough 25 that is defined between the rims 23 and is thus isolated, it can be kept from undesirably hurting the patient's skin. Further, since the two rims 23 are of the same length and both show a curved arrangement with the curve section 24, the rims 23 can comfortably and stably hold down the patient's skin without causing undesired hurting, making the present invention particularly suitable for subcutaneous stitching operations.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A suture cut-off structure for subcutaneous stitching operation, comprising a forceps and a cutter blade arranged at one end of the forceps, wherein
the forceps has an end forming a stalk-like cutter holder in which a slit is formed; and
the cutter blade forms a through hole, which comprises a relatively-wide first retention section and a relatively-narrow second retention section, the first retention section being fit over a body of the stalk-like cutter holder, the second retention section being fit into the slit of the stalk-like cutter holder so as to securely retain the cutter blade on the stalk-like cutter holder, thereby coupling the cutter blade and the stalk-like cutter holder together; the cutter blade has an end forming two rims, which correspond to each other and have substantially the same length, each of the rims having a contour forming a curve section, the rims defining a cutting trough therebetween, a cutting edge being formed in the cutting trough, whereby the suture is receivable into the cutting trough to allow the suture to be cut off through a forward movement of the cutting edge, and with the rims being positionable on and depressing down patient's skin, cutting of the suture and at the same time depressing down the patient's skin can be simultaneously done with a single hand, and the cutting edge is located inside the cutting trough of the rims and is thus isolated so that undesirably hurting the patient's skin can be avoided, and with the rims having the same length and showing the curve section, comfortably and stably holding down the patient's skin can be done without causing undesired hurting, thereby allowing efficiently cutting of suture in a subcutaneous stitching operation.

2. The suture cut-off structure for subcutaneous stitching operation according to claim 1, wherein the forceps forms an inclined step below the stalk-like cutter holder and the cutter blade forms a support section corresponding to the step, the step and the support section being in contact engagement with each other.
